(19) United States
(12) Patent Application Publication
(10) Pub. No.: US 2016/0317101 A1
(43) Pub. Date: Nov. 3, 2016

(54) ATHLETIC PERFORMANCE MONITORING DISPLAY SYSTEM AND METHOD

(71) Applicant: Yongqiang Li, Cupertino, CA (US)

(72) Inventor: Yongqiang Li, Cupertino, CA (US)

(21) Appl. No.: 14/699,640

(22) Filed: Apr. 29, 2015

(51) Int. Cl. A61B 5/00; A61B 5/0205; A63B 24/00; G06F 1/16; G06T 11/60

(52) U.S. Cl. CPC A61B 5/7445; G06F 1/163; G06F 1/1652; G06T 11/60; A63B 24/0062; A61B 5/02055; A61B 5/6804; A61B 5/6895; A63B 2220/40; A63B 2220/12; A61B 2503/10; A61B 2562/164

(57) ABSTRACT

An athletic performance monitoring display system is provided. The system includes a flexible display device having a flexible display screen, a first microprocessor, and an RF receiver. The flexible display screen has a first display area. The system further includes a monitoring device wore by a user that has a housing, a first physiological sensor, a second microprocessor, and an RF transmitter. The first physiological sensor generates a first physiological signal indicating a first physiological parameter. The second microprocessor induces the RF transmitter to transmit a first RF signal having a first binary parameter value representing the first physiological parameter. The RF receiver receives the first RF signal. The first microprocessor induces a graphical display object in the first display area to display the first physiological parameter.

### BACKGROUND

The inventor herein has recognized a need for an athletic performance monitoring display system that is attached to a least one of a clothing of a user, a body surface of the user, or an outer surface of sporting equipment utilized by the user.

### SUMMARY

An athletic performance monitoring display system in accordance with an exemplary embodiment is provided. The system includes a flexible display device having a flexible display screen, a first microprocessor, an RF receiver, a flexible housing portion, and a first adhesive member. The flexible display screen has a first side, a second side, and a first display area disposed on the first side thereof. The first microprocessor is operably coupled to the flexible display screen and the RF receiver. The flexible housing portion is coupled to a portion of the second side of the flexible display screen such that the first microprocessor and the RF receiver are disposed within an interior area defined by the second side of the flexible display screen and the flexible housing portion. The first adhesive member is coupled to an external surface of the flexible housing portion. The system further includes a monitoring device adapted to be worn by a user. The monitoring device has a housing, a first physiological sensor, a second microprocessor, and an RF transmitter. The second microprocessor is operably coupled to the first physiological sensor and the RF transmitter. The first physiological sensor is adapted to generate a first physiological signal indicating a magnitude of a first physiological parameter of the user at a first time. The second microprocessor is programmed to generate a first control signal to induce the RF transmitter to transmit a first RF signal having a first binary parameter value representing the magnitude of the first physiological parameter of the user at the first time, based on the first physiological signal. The RF receiver of the flexible display device is adapted to receive the first RF signal and to send the first binary parameter value therein to the first microprocessor. The first microprocessor is programmed to generate a second control signal to induce a graphical display object in the first display area to display the magnitude of the first physiological parameter at the first time, based on the first binary parameter value.

A method for displaying information associated with a user in accordance with another exemplary embodiment is provided. The method includes attaching a flexible display device to the user. The flexible display device has a flexible display screen, a first microprocessor, an RF receiver, a flexible housing portion, and a first adhesive member. The flexible display screen has a first side, a second side, and a first disposed on the first side thereof. The first microprocessor is operably coupled to the flexible display screen and the RF receiver. The flexible housing portion is coupled to the second side of the flexible display screen such that the first microprocessor and the RF receiver are disposed within an interior area defined by the second side of the flexible display screen and the flexible housing portion. The first adhesive member is coupled to an external surface of the flexible housing portion and to the user. The method further includes attaching a monitoring device to the user. The monitoring device has a housing, a first physiological sensor, a second microprocessor, and an RF transmitter. The second microprocessor is operably coupled to the first physiological sensor and the RF transmitter. The method further includes generating a first physiological signal indicating a magnitude of a first physiological parameter of the user at a first time utilizing the first physiological sensor. The method further includes generating a first control signal from the second microprocessor to induce the RF transmitter to transmit a first RF signal having a first binary parameter value representing the magnitude of the first physiological parameter of the user at the first time, based on the first physiological signal. The method further includes receiving the first RF signal at the RF receiver of the flexible display device and sending the first binary parameter value to the first microprocessor. The method further includes generating a second control signal from the first microprocessor to induce a graphical display object in the first display area to display the magnitude of the first physiological parameter at the first time, based on the first binary parameter value.

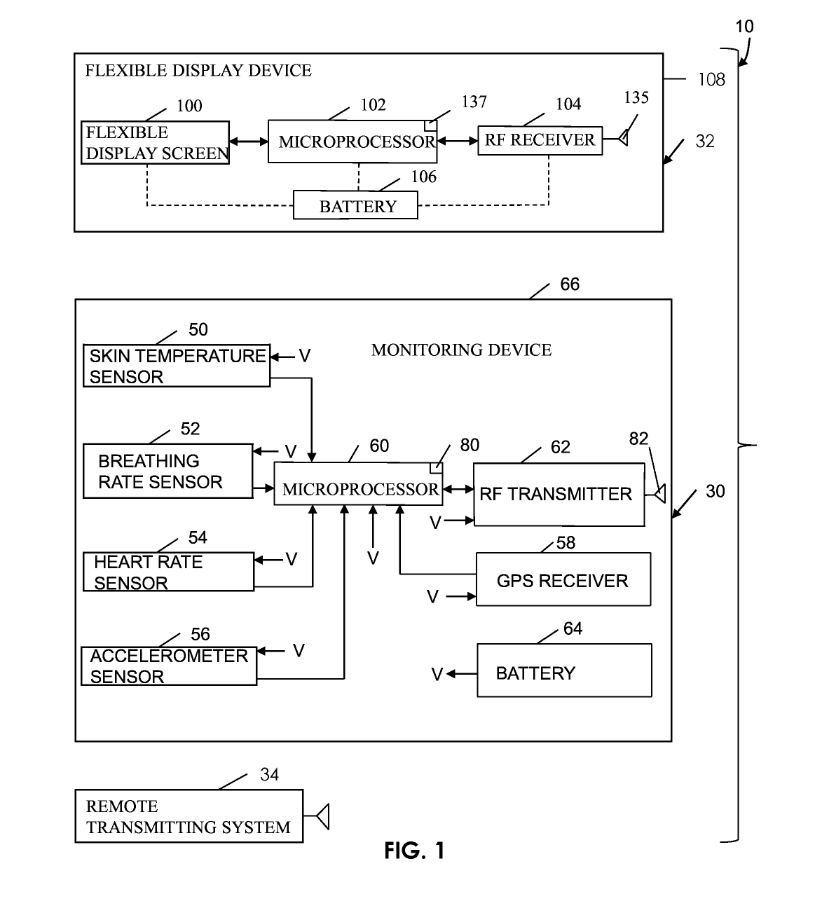

FIG. 1 is a schematic of an athletic performance monitoring display system in accordance with an exemplary embodiment;

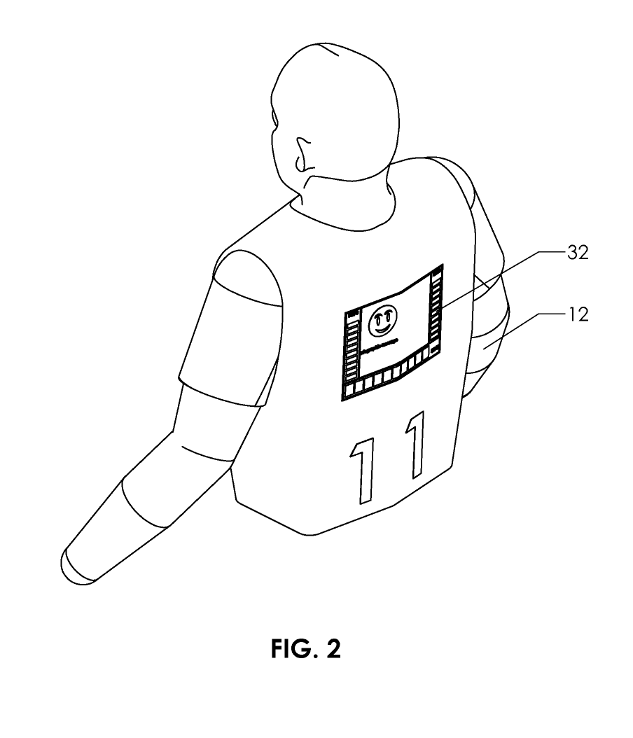

FIG. 2 is a schematic of a user wearing a shirt having a flexible display device of the athletic performance monitoring display system of FIG. 1 coupled to the shirt;

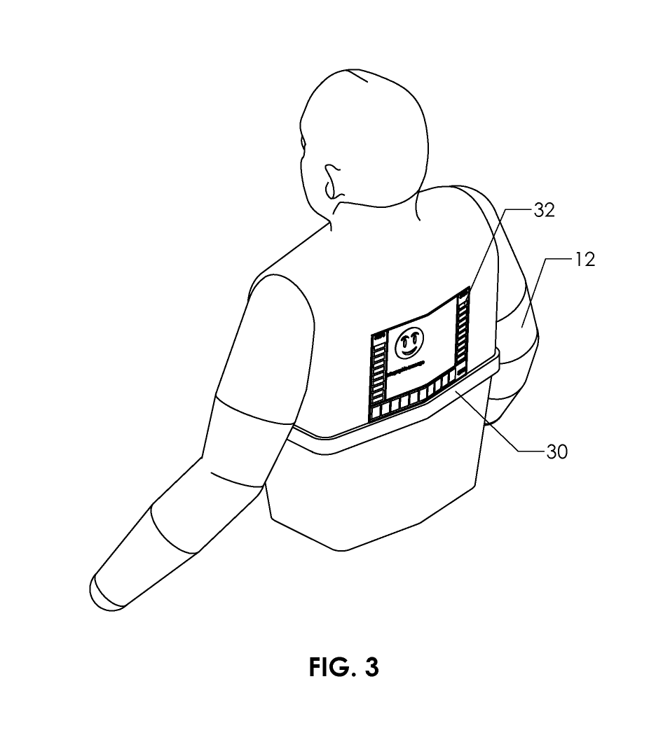

FIG. 3 is a schematic of a user having the flexible display device of FIG. 2 and a monitoring device of the athletic performance monitoring display system of FIG. 1 coupled to the body of the user;

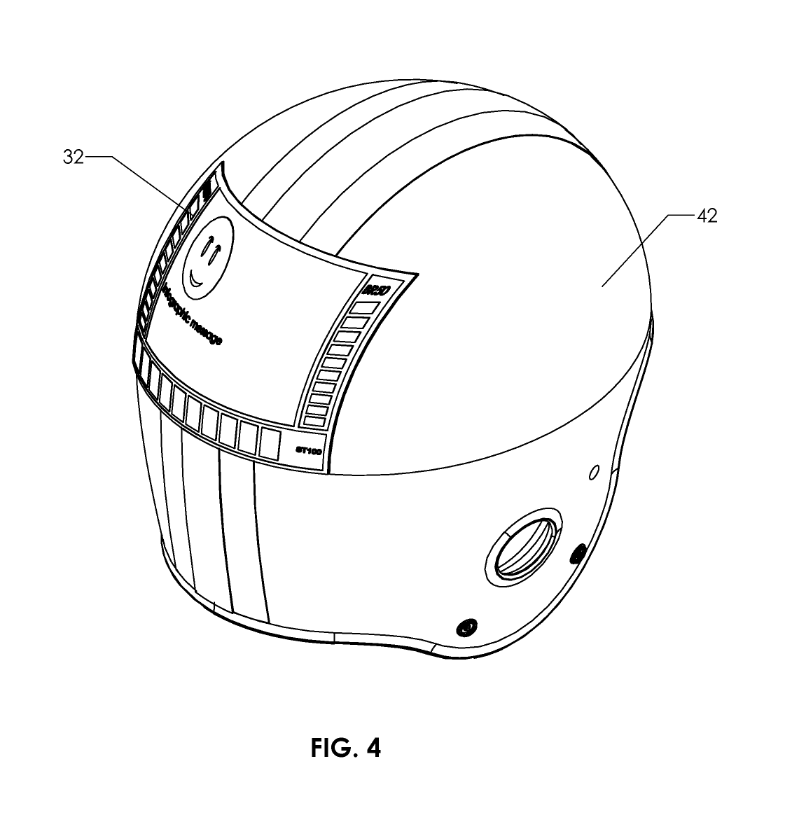

FIG. 4 is a schematic of a helmet having the flexible display device of FIG. 21 coupled thereto;

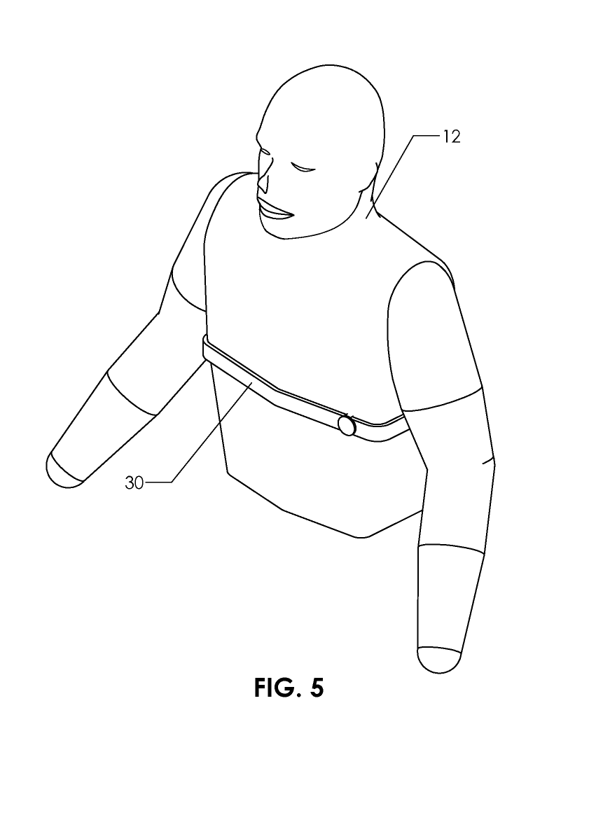

FIG. 5 is a schematic of the user wearing the monitoring device of FIG. 3;

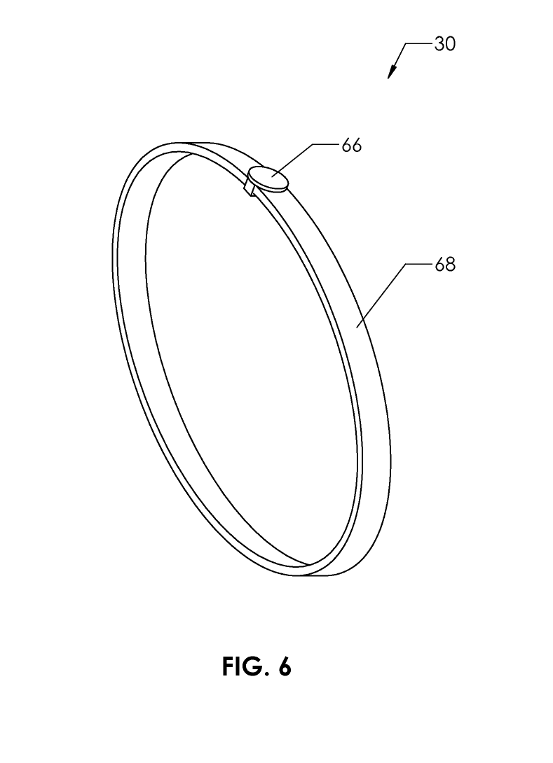

FIG. 6 is an enlarged schematic of the monitoring device of FIG. 5;

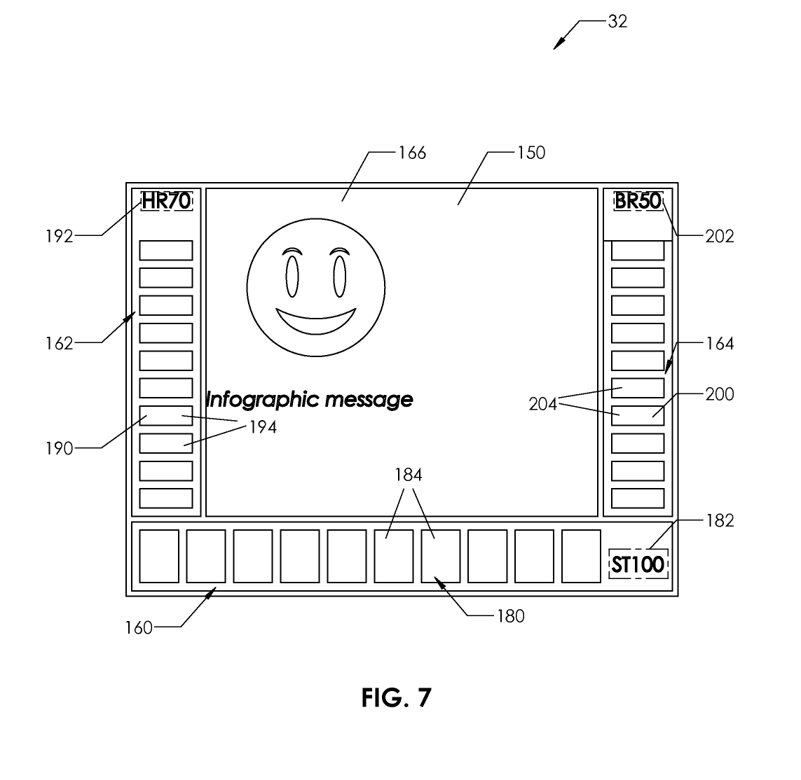

FIG. 7 is a schematic of a front side of the flexible display device of FIG. 2 displaying a first set of information associated with the user;

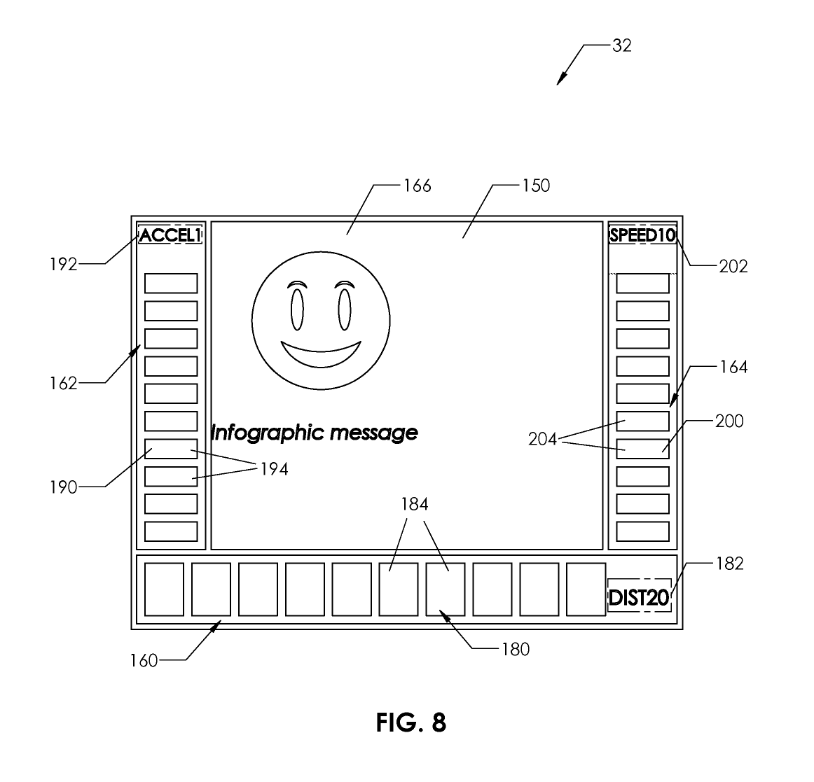

FIG. 8 is another schematic of the front side of the flexible display device of FIG. 2 displaying a second set of information associated with the user;

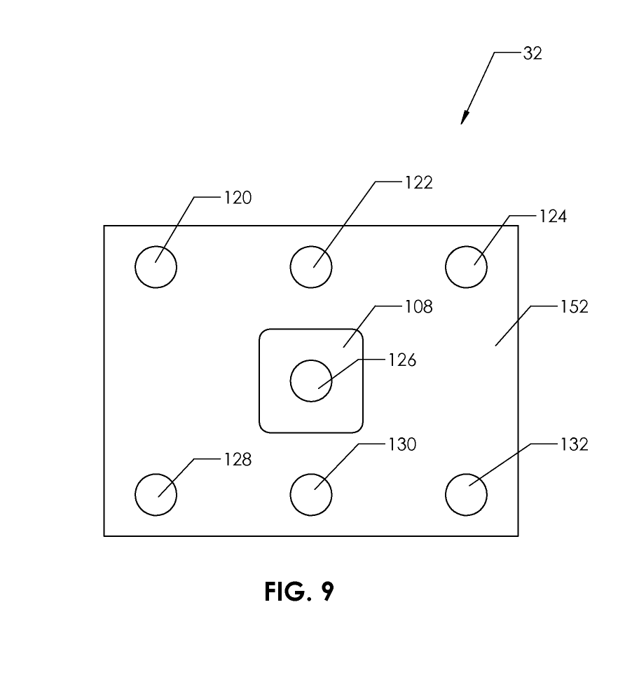

FIG. 9 is a schematic of a rear side of the flexible display device of FIG. 2;

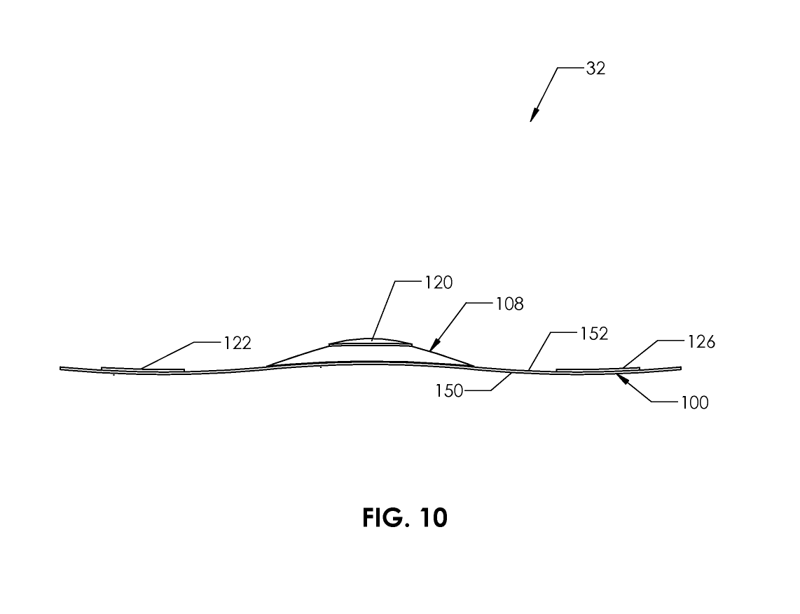

FIG. 10 is a side view of the flexible display device of FIG. 2;

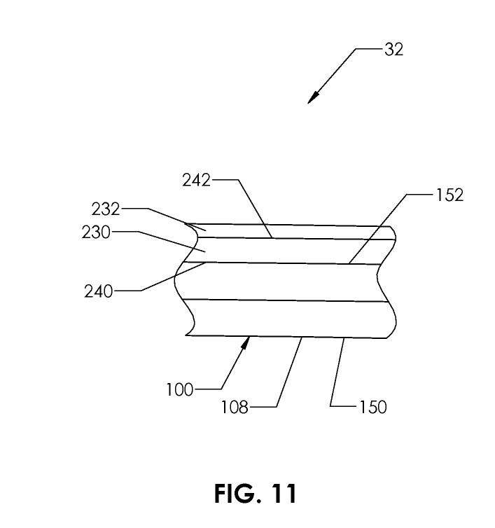

FIG. 11 is a cross-sectional view of a portion of the flexible display device of FIG. 2; and

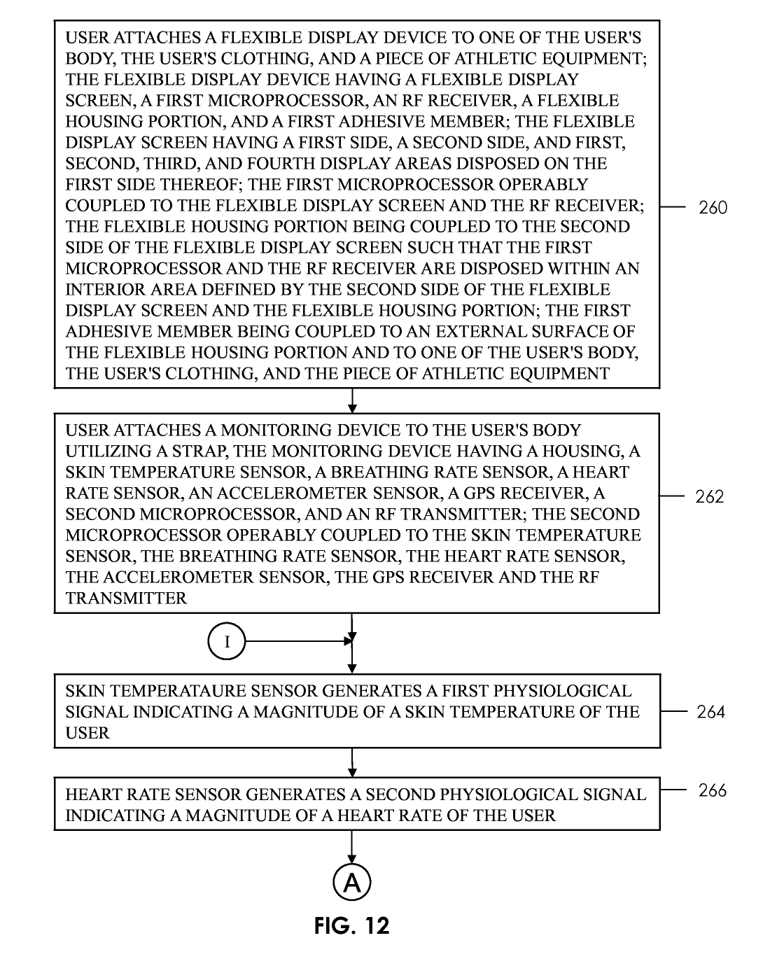

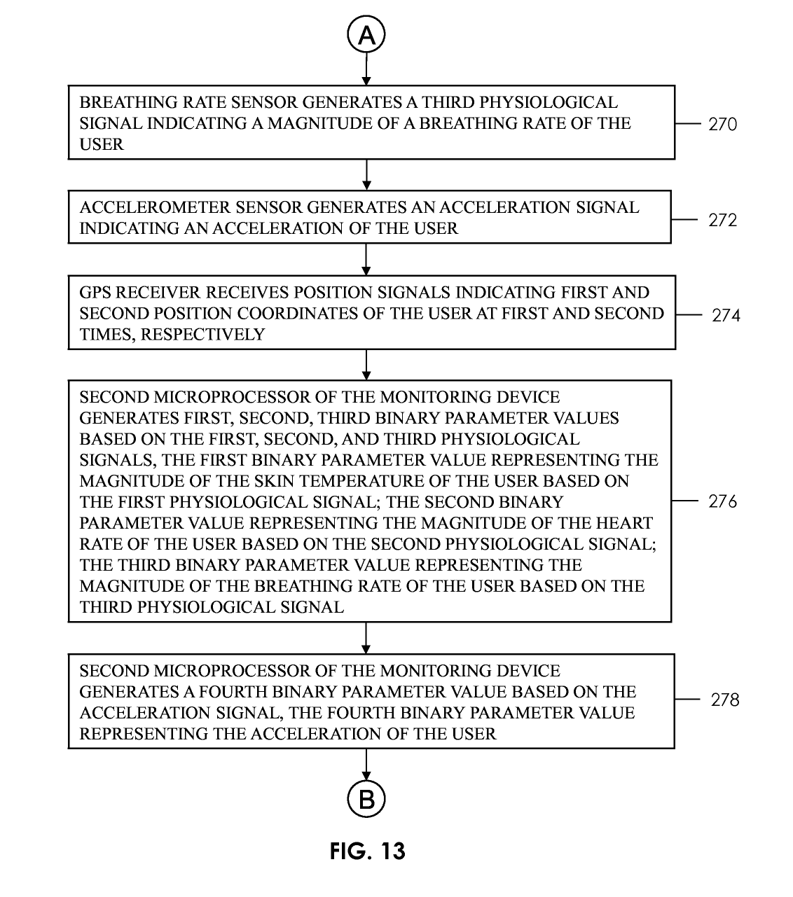

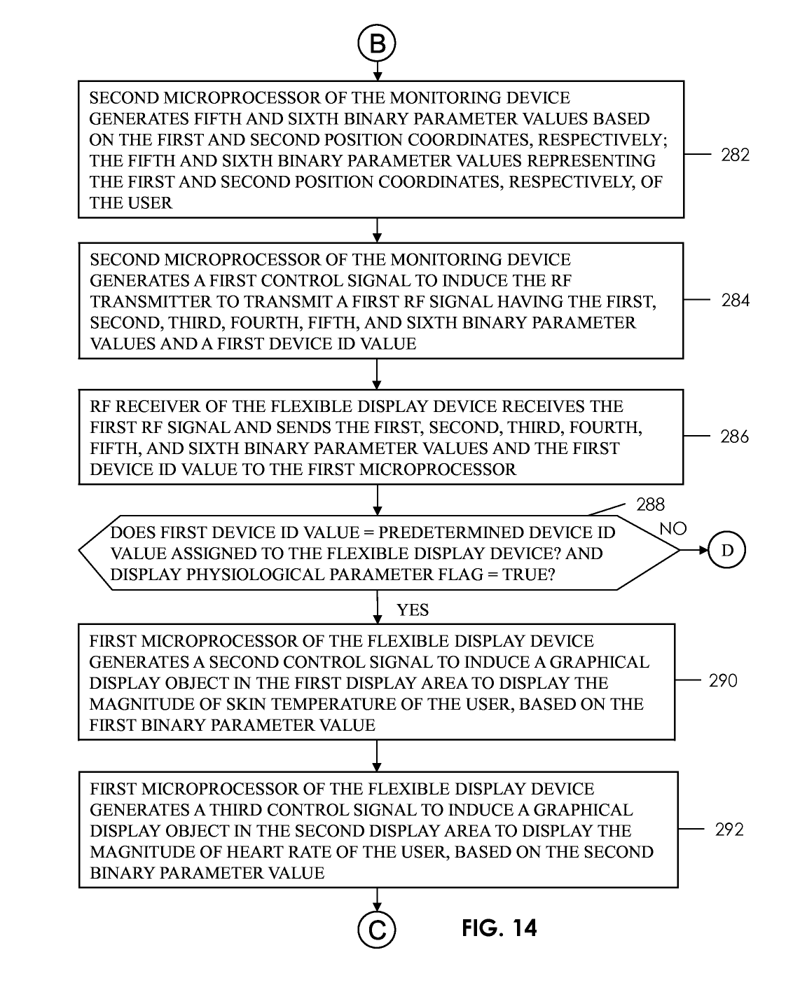

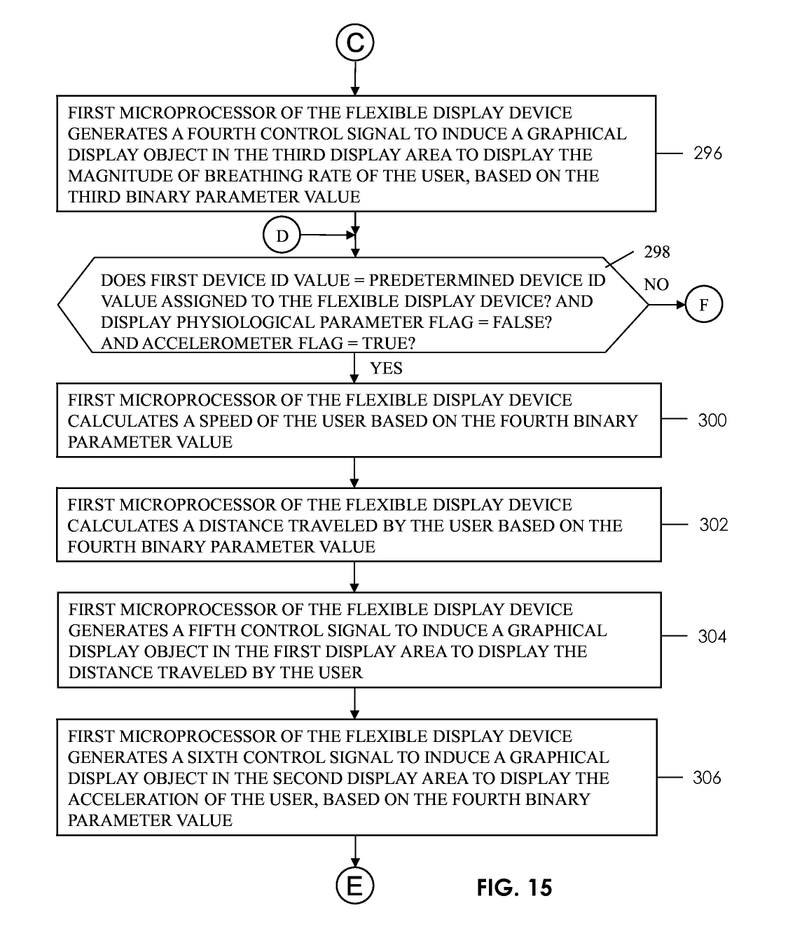

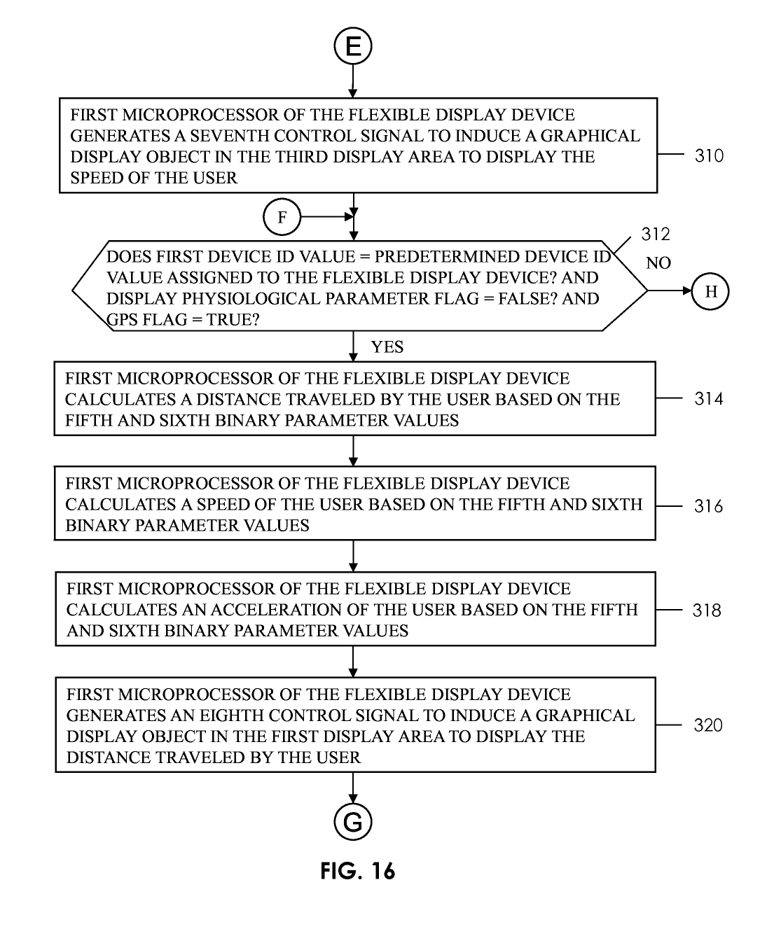

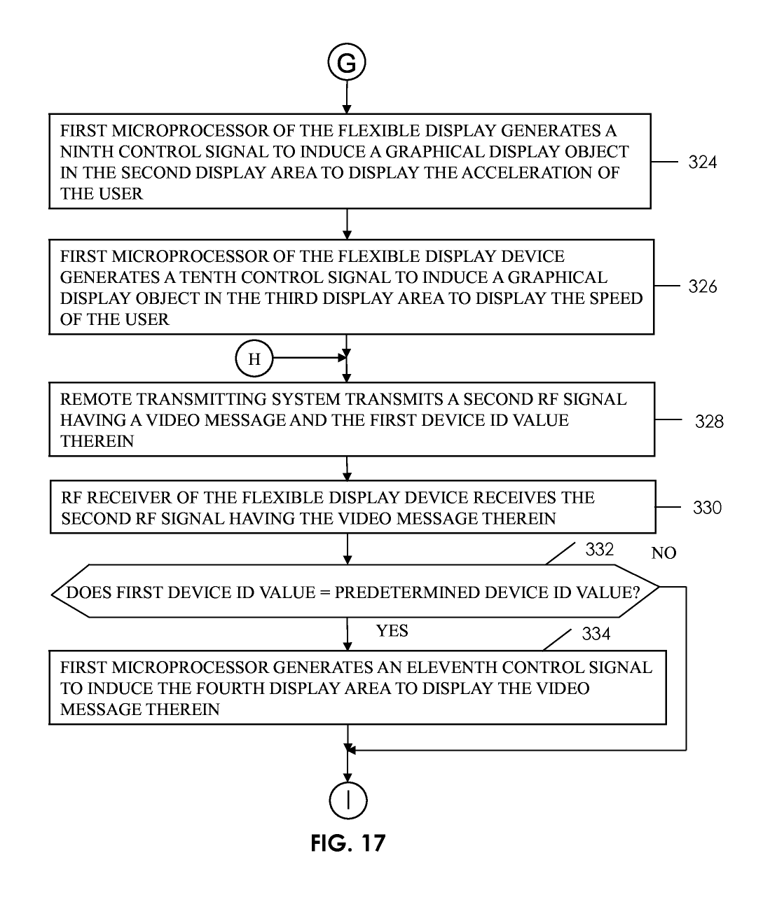

FIGS. 12-17 are flowcharts of a method for displaying information associated with a user in accordance with another exemplary embodiment.

### DETAILED DESCRIPTION

Referring to FIGS. 1 and 2, an athletic performance monitoring display system 10 in accordance with an exemplary embodiment is provided. The system 10 includes a monitoring device 30, a flexible display device 32, and a remote transmitting system 34. An advantage of the system 10 is that the system 10 utilizes the monitoring device 32 to monitor physiological parameters and non-physiological parameters associated with a user 12 and then transmits the parameter information to the flexible display device 32 which displays the information. Thus, viewers of an athletic performance by the user 12 can view the physiological parameter information and the non-physiological information associated with the user 12.

Referring to FIGS. 1, 3 and 6, the monitoring device 30 is adapted to be worn by the user 12. The monitoring device includes a skin temperature sensor 50, a breathing rate sensor 52, a heart rate sensor 54, an accelerometer sensor 56, a GPS receiver 58, a microprocessor 60, an RF transmitter 62, a battery 64, a housing 66, and a strap 68.

The housing 66 is adapted to hold the skin temperature sensor 50, the breathing rate sensor 52, the heart rate sensor 54, the accelerometer sensor 56, the GPS receiver 58, the microprocessor 60, the RF transmitter 62, and the battery 64 therein. The strap 68 is coupled to the housing 66 and is provided to hold the housing 66 against the body of the user 12. In an exemplary embodiment, the strap 68 is a ring-shaped elastic strap adapted to hold the housing 66 directly on and against a chest of the user 12.

The skin temperature sensor 50, the breathing rate sensor 52, and the heart rate sensor 54 are each physiological sensors adapted to generate physiological signals indicating a magnitude of physiological parameters of the user. In particular, the skin temperature sensor 50 generates a first physiological signal (e.g., a temperature level signal) indicating a magnitude of a skin temperature of the user 12 that is received by the microprocessor 60. Further, the heart rate sensor 54 generates a second physiological signal (e.g., heart rate signal) indicating a magnitude of a heart rate of the user 12 that is received by the microprocessor 60. Further, the breathing rate sensor 52 generates a third physiological signal (e.g., breathing rate signal) indicating a magnitude of a breathing rate of the user 12 that is received by the microprocessor 60.

The accelerometer sensor 56 is adapted to generate an acceleration signal indicating an acceleration of the user 12 that is received by the microprocessor 60.

The GPS receiver 58 is adapted to receive position signals from at least three GPS satellites. The GPS receiver 58 determines first and second position coordinates of the user 12 at first and second times, respectively, based on the position signals from the GPS satellites, and sends the first and second position coordinates of the user 12 to the microprocessor 60.

The microprocessor 60 is operably coupled to the skin temperature sensor 50, the breathing rate sensor 52, the heart rate sensor 54, the accelerometer sensor 56, the GPS receiver 58, the RF transmitter 62, and the battery 64. The microprocessor 60 is adapted to receive the first, second, and third physiological signals from the skin temperature sensor 50, the heart rate sensor 54, and the breathing rate sensor 52, respectively. The microprocessor 60 is programmed to generate first, second, third binary parameter values based on the first, second, and third physiological signals, respectively. The first binary parameter value represents the magnitude of the skin temperature of the user 12 based on the first physiological signal. The second binary parameter value represents the magnitude of the heart rate of the user 12 based on the second physiological signal. The third binary parameter value represents the magnitude of the breathing rate of the user 12 based on the third physiological signal.

The microprocessor 60 is further adapted to receive the acceleration signal from the accelerometer sensor 56, and the first and second position coordinates from the GPS receiver 58. The microprocessor 60 is programmed to generate a fourth binary parameter value based on the acceleration signal. The fourth binary parameter value represents the acceleration of the user 12. Further, the microprocessor 60 is programmed to generate fifth and sixth binary parameter values based on the first and second position coordinates, respectively. The fifth and sixth binary parameter values represent the first and second position coordinates, respectively, of the user 12 at the first and second times, respectively.

The microprocessor 60 is further programmed to generate a first control signal to induce the RF transmitter 62 to transmit a first RF signal having the first, second, third, fourth, fifth, and sixth binary parameter values and a first device ID value. The first device ID value is associated with the flexible display device 32. The microprocessor 60 includes a memory device 80 configured to hold software instructions and data for implementing the method steps associated with the microprocessor 60 described herein.

The battery 64 is adapted to provide an operational voltage to the skin temperature sensor 50, the breathing rate sensor 52, the heart rate sensor 54, the accelerometer sensor 56, the microprocessor 60, the GPS receiver 58, and the RF transmitter 62.

Referring to FIGS. 1 and 7-11, the flexible display device 32 is provided to display physiological information and non-physiological information associated with user 12 thereon. The flexible display device 32 includes a flexible display screen 100, a microprocessor 102, an RF receiver 104, a battery 106, a flexible housing portion 108, and adhesive members 120, 122, 124, 126, 128, 130, 132 (shown in FIG. 9). The flexible display screen 100 has a first side 150 (shown in FIG. 11), a second side 152, and display areas 160, 162, 164, 166 disposed on the first side 150.

Referring to FIG. 1, the RF receiver 104 is adapted to receive the first RF signal from the RF transmitter 62 of the monitoring device 30, and to send the first, second, third, fourth, fifth, and sixth binary parameter values and the first device ID value to the microprocessor 102. The RF receiver 104 includes an antenna 135 configured to receive RF signals. The RF receiver 104 is further configured to receive a second RF signal having a video message therein from the remote transmitting system 34.

The microprocessor 102 is operably coupled to the flexible display screen 100, the RF receiver 104, and the battery 106. The microprocessor 102 is programmed to receive the first, second, third, fourth, fifth, and sixth binary parameter values and the first device ID value from the RF receiver 104, and to generate control signals for inducing the flexible display screen 100 to display information associated with the first, second, third, fourth, fifth, and sixth binary parameter values thereon, as will be described in greater detail below. The microprocessor 102 is further programmed to receive a video message from the RF receiver 104 and to generate a control signal for inducing the flexible display screen 100 to display the video message thereon, as will be explained in greater detail below. The microprocessor 102 includes a memory device 137 configured to hold software instructions and data for implementing the method steps associated with the microprocessor 102 described herein.

Referring to FIG. 7, the flexible display screen 100 has a first operational mode in which the display areas 160, 162, 164 display physiological information associated with the user 12, and the display area 166 displays a video message therein.

The display area 160 includes a graphical display object 180 and a textual area 182 that displays information therein based on first and second control signals, respectively, from the microprocessor 102. In a first operational mode of the flexible display screen 100, the graphical display object 180 is adapted to display a skin temperature of the user 12. In an exemplary embodiment, the graphical display object 180 comprises a bar graph having a plurality of rectangular segments 184 disposed in a column and separated from one another. A number of the rectangular segments of the plurality rectangular segments 184 having a solid dark color represent the magnitude of the skin temperature of the user 12. Further, the display area 160 includes a textual display area 182 adapted to display text (e.g., ST) identifying the first physiological parameter (e.g., skin temperature) having a decimal number indicating a magnitude of the skin temperature of the user 12. For example, the textual display area 182 can display “ST100” indicating a skin temperature of 100° F. for the user 12.

The display area 162 includes a graphical display object 190 and a textual area 192 that displays information therein based on first and second control signals, respectively, from the microprocessor 102. In the first operational mode of the flexible display screen 100, the display area 162 displays physiological parameter information associated with the user 12. In particular, the graphical display object 190 is adapted to display a heart rate of the user 12. In an exemplary embodiment, the graphical display object 190 comprises a bar graph having a plurality of rectangular segments 194 disposed in a column and separated from one another. A number of the rectangular segments of the plurality rectangular segments 194 having a solid dark color represent the magnitude of the heart rate of the user 12. Further, the display area 162 includes a textual display area 192 adapted to display text (e.g., HR) identifying the second physiological parameter (e.g., heart rate) having a decimal number indicating a magnitude of the heart rate of the user 12. For example, the textual display area 192 can display “HR70” indicating a heart rate of 70 beats per minute for the user 12.

The display area 164 includes a graphical display object 200 and a textual area 202 that displays information therein based on first and second control signals, respectively, from the microprocessor 102. In the first operational mode of the flexible display screen 100, the display area 164 displays physiological parameter information associated with the user 12. In particular, the graphical display object 200 is adapted to display a breathing rate of the user 12. In an exemplary embodiment, the graphical display object 200 comprises a bar graph having a plurality of rectangular segments 204 disposed in a column and separated from one another. A number of the rectangular segments of the plurality rectangular segments 204 having a solid dark color represent the magnitude of the breathing rate of the user 12. Further, the display area 164 includes a textual display area 202 adapted to display text (e.g., BR) identifying the third physiological parameter (e.g., breathing rate) having a decimal number indicating a magnitude of the breathing rate of the user 12. For example, the textual display area 202 can display “BR50” indicating a breathing rate of 50 breaths per minute for the user 12.

Referring to FIG. 8, the flexible display screen 100 has a second operational mode in which the display areas 160, 162, 164 display non-physiological information associated with the user 12, and the display area 166 displays a video message therein.

In the second operational mode of the flexible display screen 100, the graphical display object 180 is adapted to display a distance moved by the user 12. A number of the rectangular segments of the plurality rectangular segments 184 having a solid dark color represent the distance moved by the user 12. Further, the display area 160 includes the textual display area 182 adapted to display text (e.g., DIST) identifying the non-physiological parameter (e.g., distance) having a decimal number indicating a distance moved by the user 12. For example, the textual display area 182 can display “DIST20” indicating the user 12 moved a distance of 20 feet.

In the second operational mode of the flexible display screen 100, the graphical display object 190 is adapted to display an acceleration of the user 12. A number of the rectangular segments of the plurality rectangular segments 194 having a solid dark color represent the acceleration of the user 12. Further, the display area 162 includes the textual display area 192 adapted to display text (e.g., ACCEL) identifying the non-physiological parameter (e.g., acceleration) having a decimal number indicating an acceleration of the user 12. For example, the textual display area 192 can display “ACCEL1” indicating an acceleration of 1 g (gravitational acceleration) for the user 12.

In the second operational mode of the flexible display screen 100, the display area 164 displays non-physiological parameter information associated with the user 12. In particular, the graphical display object 200 is adapted to display a speed of the user 12. A number of the rectangular segments of the plurality rectangular segments 204 having a solid dark color represent the speed of the user 12. Further, the display area 164 includes a textual display area 202 adapted to display text (e.g., SPEED) identifying the non-physiological parameter (e.g., speed) having a decimal number indicating the speed of the user 12. For example, the textual display area 202 can display “SPEED10” indicating a speed of 10 miles per hour for the user 12.

Referring to FIG. 1, the battery 106 is adapted to provide an operational voltage to the flexible display screen 100, the microprocessor 102, and the RF receiver 104.

Referring to FIGS. 1 and 10, the flexible housing portion 108 is coupled to a portion of the second side 152 of the flexible display screen 100 such that the first microprocessor 102, the RF receiver 104, and the battery 106 are disposed within an interior area defined by the second side 152 of the flexible display screen 100 and the flexible housing portion 108. In an exemplary embodiment, the flexible housing portion 108 is constructed of a thermoplastic material.

Referring to FIGS. 9 and 11, the adhesive members 120, 122, 124, 126, 128, 130, 132 are coupled to an external surface of the flexible housing portion 108. The adhesive members 120-132 are provided to removably couple the flexible display device 32 directly to at least one of a clothing (shown in FIG. 2) of the user 12, a body surface (shown in FIG. 3) of the user 12, and an outer surface of sporting equipment (shown in FIG. 4) utilized by the user 12. Since the structure of the adhesive members 120-132 are identical to one another, only the structure of the adhesive member 126 will be explained in greater detail below.

The adhesive member 126 includes a double-sided adhesive layer 230 and a peelable layer 232. The adhesive layer 230 has first and second surfaces 240, 242. The first surface 240 is disposed on and adhered to the external surface of the flexible housing portion 108. The peelable layer 232 is removably disposed on the second surface of the adhesive layer 230 by the user 12. When the peelable layer 232 is removed from the adhesive layer 230, the adhesive layer 230 can be removably attached to at least one of the clothing of the user 12, the body surface of the user 12, and the outer surface of sporting equipment (e.g., the helmet 42 shown in FIG. 4) utilized by the user 12.

Further, the adhesive members 120, 122, 124, 128, 130, 132 are disposed on and coupled to a second side of the flexible display device 32 and are removably attached to at least one of the clothing of the user 12, the body surface of the user 12, and the outer surface of sporting equipment (e.g., the helmet 42 shown in FIG. 4) utilized by the user 12.

Referring to FIG. 1, the remote transmitting system 34 is adapted to transmit an RF signal having video information, or static text information, or static graphical information therein to the flexible display device 32. The video information can be either a commercial video message or a non-commercial video message.

Referring to FIGS. 1 and 12-17, a flowchart of a method for displaying information associated with the user 12 utilizing the athletic performance monitoring display system 10 in accordance with another exemplary embodiment will be explained. It is noted that although the steps of the exemplary method is described in a specific order, the steps could be performed in an alternative order.

At step 260, the user 12 attaches the flexible display device 32 to one of the user's body, the user's clothing, and a piece of athletic equipment. The flexible display device 32 has the flexible display screen 100, the microprocessor 102, the RF receiver 104, the flexible housing portion 108, and the adhesive member 126. The flexible display screen 100 has the first side 150, the second side 152, and display areas 160, 162, 164, 166 disposed on the first side 150 thereof. The microprocessor 102 is operably coupled to the flexible display screen 100 and the RF receiver 104. The flexible housing portion 108 is coupled to the second side 152 of the flexible display screen 100 such that the microprocessor 102 and the RF receiver 104 are disposed within an interior area defined by the second side 152 of the flexible display screen 100 and the flexible housing portion 108. The adhesive member 126 is coupled to an external surface of the flexible housing portion 108 and to one of the user's body, the user's clothing, and the piece of athletic equipment. After step 260, the method advances to step 262.

At step 262, the user 12 attaches the monitoring device 30 to the user's body utilizing the strap 68. The monitoring device 30 includes the housing 66, the skin temperature sensor 50, the breathing rate sensor 52, the heart rate sensor 54, the accelerometer sensor 56, the GPS receiver 58, the microprocessor 60, and the RF transmitter 62. The microprocessor 60 is operably coupled to the skin temperature sensor 50, the breathing rate sensor 52, the heart rate sensor 54, the accelerometer sensor 56, the GPS receiver 58 and the RF transmitter 62. After step 262, the method advances to step 264.

At step 264, the skin temperature sensor 50 generates a first physiological signal indicating a magnitude of a skin temperature of the user 12. After step 264, the method advances to step 266.

At step 266, the heart rate sensor 54 generates a second physiological signal indicating a magnitude of a heart rate of the user 12. After step 266, the method advances to step 270.

At step 270, the breathing rate sensor 52 generates a third physiological signal indicating a magnitude of a breathing rate of the user 12. After step 270, the method advances to step 272.

At step 272, the accelerometer sensor 56 generates an acceleration signal indicating an acceleration of the user 12. After step 272, the method advances to step 274.

At step 274, the GPS receiver 58 receives position signals indicating first and second position coordinates of the user 12 at first and second times, respectively, from GPS satellites. After step 274, the method advances to step 276.

At step 276, the microprocessor 60 of the monitoring device 30 generates first, second, third binary parameter values based on the first, second, and third physiological signals. The first binary parameter value represents the magnitude of the skin temperature of the user 12 based on the first physiological signal. The second binary parameter value represents the magnitude of the heart rate of the user 12 based on the second physiological signal. Further, the third binary parameter value represents the magnitude of the breathing rate of the user 12 based on the third physiological signal. After step 276, the method advances to step 278.

At step 278, the microprocessor 60 of the monitoring device 30 generates a fourth binary parameter value based on the acceleration signal. The fourth binary parameter value represents the acceleration of the user 12. After step 278, the method advances to step 282.

At step 282, the microprocessor 60 of the monitoring device 30 generates fifth and sixth binary parameter values based on the first and second position coordinates, respectively. The fifth and sixth binary parameter values represent the first and second position coordinates, respectively, of the user 12, at the first and second times, respectively. After step 282, the method advances to step 284.

At step 284, the microprocessor 60 of the monitoring device 30 generates a first control signal to induce the RF transmitter 62 to transmit a first RF signal having the first, second, third, fourth, fifth, and sixth binary parameter values and a first device ID value. After step 284, the method advances to step 286.

At step 286, the RF receiver 104 of the flexible display device 32 receives the first RF signal and sends the first, second, third, fourth, fifth, and sixth binary parameter values and the first device ID value to the microprocessor 102. After step 286, the method advances to step 288.

At step 288, the microprocessor 102 makes a determination as to whether the first device ID value equals a predetermined device ID value assigned to the flexible display device 32, and whether a display physiological parameter flag is equal to true. If the value of step 288 equals “yes”, the method advances to step 290. Otherwise, the method advances to step 298.

At step 290, the microprocessor 102 of the flexible display device 32 generates a second control signal to induce the graphical display object 180 in the first display area 160 to display the magnitude of skin temperature of the user 12, based on the first binary parameter value. After step 290, the method advances to step 292.

At step 292, the microprocessor 102 of the flexible display device 32 generates a third control signal to induce the graphical display object 190 in the second display area 162 to display the magnitude of heart rate of the user 12, based on the second binary parameter value. After step 292, the method advances to step 296.

At step 296, the microprocessor 102 of the flexible display device 32 generates a fourth control signal to induce the graphical display object 200 in the third display area 164 to display the magnitude of breathing rate of the user 12, based on the third binary parameter value. After step 296, the method advances the step 298.

At step 298, the microprocessor 102 makes a determination as to whether the first device ID value is equal to the predetermined device ID value assigned to the flexible display device 32, and whether the display physiological parameter flag is equal to false, and whether the accelerometer flag is equal to true. If the value of step 298 equals “true”, the method advances to step 300. Otherwise, the method advances to step 312.

At step 300, the microprocessor 102 of the flexible display device 32 calculates a speed of the user 12 based on the fourth binary parameter value. In particular, the microprocessor 102 can mathematically integrate the fourth binary parameter value over a time interval to obtain the speed of the user 12. After step 300, the method advances to step 302.

At step 302, the microprocessor 102 of the flexible display device 32 calculates a distance traveled by the user 12 based on the fourth binary parameter value. In particular, the microprocessor 102 can perform a double mathematical integration of the fourth binary parameter value over a time interval to obtain the distance traveled by the user 12. After step 302, the method advances to step 304.

At step 304, the microprocessor 102 of the flexible display device 32 generates a fifth control signal to induce the graphical display object 180 in the first display area 160 to display the distance traveled by the user 12. After step 304, the method advances to step 306.

At step 306, the microprocessor 102 of the flexible display device 32 generates a sixth control signal to induce the graphical display object 190 in the second display area 162 to display the acceleration of the user 12, based on the fourth binary parameter value. After step 306, the method advances to step 310.

At step 310, the microprocessor 102 of the flexible display device 32 generates a seventh control signal to induce the graphical display object 200 in the third display area 164 to display the speed of the user 12. After step 310, the method advances to step 312.

At step 312, the microprocessor 102 makes a determination as to whether the first device ID value is equal to the predetermined device ID value assigned to the flexible display device 32, and whether the display physiological parameter flag is equal to false, and whether the GPS flag is equal to true. If the value of step 312 equals “yes”, the method advances to step 314. Otherwise, the method advances to step 328.

At step 314, the microprocessor 102 of the flexible display device 32 calculates a distance traveled by the user 12 based on the fifth and sixth binary parameter values. After step 314, the method advances to step 316.

At step 316, the microprocessor 102 of the flexible display device 32 calculates a speed of the user 12 based on the fifth and sixth binary parameter values. In particular, the microprocessor 102 can mathematically calculate the speed of the user 12 based on the difference of the fifth and sixth binary parameter values divided by a time interval. After step 316, the method advances to step 318.

At step 318, the microprocessor 102 of the flexible display device 32 calculates an acceleration of the user 12 based on the fifth and sixth binary parameter values. In particular, the microprocessor 102 can mathematically calculate the acceleration of the user 12 based on the difference of the fifth and sixth binary parameter values divided by a time interval squared. After step 318, the method advances to step 320.

At step 320, the microprocessor 102 of the flexible display device 32 generates an eighth control signal to induce the graphical display object 180 in the first display area 160 to display the distance traveled by the user 12. After step 320, the method advances to step 324.

At step 324, the microprocessor 102 of the flexible display device 32 generates a ninth control signal to induce the graphical display object 190 in the second display area 162 to display the acceleration of the user 12. After step 324, the method advances to step 326.

At step 326, the microprocessor 102 of the flexible display device 32 generates a tenth control signal to induce the graphical display object 200 in the third display area 164 to display the speed of the user 12. After step 326, the method advances to step 328.

At step 328, the remote transmitting system 34 transmits a second RF signal having a video message and the first device IDS value therein. After step 328, the method advances to step 330.

At step 330, the RF receiver 104 of the flexible display device 32 receives the second RF signal having the video message therein. After step 330, the method advances to step 332.

At step 332, the microprocessor 102 makes a determination as to whether the first device ID value is equal to the predetermined device ID value. If the value of step 332 equals “yes”, the method advances to step 334. Otherwise, the method returns to step 264.

At step 334, the microprocessor 102 generates an eleventh control signal to induce the fourth display area 150 to display the video message therein. After step 334, the method returns to step 264.

The above-described method can be at least partially embodied in the form of one or more memory devices or computer readable media having computer-executable instructions for practicing the methods. The memory devices can comprise one or more of the following: hard drives, RAM memory, flash memory, and other computer-readable media known to those skilled in the art; wherein, when the computer-executable instructions are loaded into and executed by one or more computers or microprocessors, the one or more computers or microprocessors become an apparatus programmed to practice the associated steps of the method.

The athletic performance monitoring display system 10 and the method described herein provide a substantial advantage over other systems. In particular, the system 10 utilizes the monitoring device 32 to monitor physiological parameters and non-physiological parameters associated with the user and then transmits the parameter information to the flexible display device 32 which displays the information. Thus, viewers of an athletic performance by the user 12 can view the physiological parameter information and the non-physiological parameter information associated with the user.

While the claimed invention has been described in detail in connection with only a limited number of embodiments, it should be readily understood that the invention is not limited to such disclosed embodiments. Rather, the claimed invention can be modified to incorporate any number of variations, alterations, substitutions or equivalent arrangements not heretofore described, but which are commensurate with the spirit and scope of the invention. Additionally, while various embodiments of the claimed invention have been described, it is to be understood that aspects of the invention may include only some of the described embodiments. Accordingly, the claimed invention is not to be seen as limited by the foregoing description.

What is claimed is:

1. An athletic performance monitoring display system, comprising:
a flexible display device having a flexible display screen, a first microprocessor, an RF receiver, a flexible housing portion, and a first adhesive member;
the flexible display screen having a first side, a second side, and a first display area disposed on the first side thereof;
the first microprocessor operably coupled to the flexible display screen and the RF receiver;
the flexible housing portion being coupled to a portion of the second side of the flexible display screen such that the first microprocessor and the RF receiver are disposed within an interior area defined by the second side of the flexible display screen and the flexible housing portion; and
the first adhesive member being coupled to an external surface of the flexible housing portion;
a monitoring device adapted to be worn by a user, the monitoring device having a housing, a first physiological sensor, a second microprocessor, and an RF transmitter; the second microprocessor operably coupled to the first physiological sensor and the RF transmitter; the first physiological sensor being adapted to generate a first physiological signal indicating a magnitude of a first physiological parameter of the user at a first time, the second microprocessor programmed to generate a first control signal to induce the RF transmitter to transmit a first RF signal having a first binary parameter value representing the magnitude of the first physiological parameter of the user at the first time, based on the first physiological signal; and
the RF receiver of the flexible display device being adapted to receive the first RF signal and to send the first binary parameter value therein to the first microprocessor, the first microprocessor programmed to generate a second control signal to induce a graphical display object in the first display area to display the magnitude of the first physiological parameter at the first time, based on the first binary parameter value.

2. The athletic performance monitoring display system of claim 1, wherein the first adhesive member comprises an adhesive layer and a peelable layer, the adhesive layer having first and second surfaces, the first surface being disposed on and adhered to the external surface of the flexible housing portion, the peelable layer being removably disposed on the second surface of the adhesive layer.

3. The athletic performance monitoring display system of claim 1, wherein the flexible display device further includes second and third adhesive members, the second and third adhesive members being coupled to the second side of the flexible display device and being spaced apart from one another, the flexible display device is attachable to at least one of a clothing of the user, a body surface of the user, and an outer surface of sporting equipment utilized by the user, utilizing the first, second, and third adhesive members.

4. The athletic performance monitoring display system of claim 1, wherein the monitoring device further includes a second physiological sensor, the second microprocessor further operably coupled to the second physiological sensor; the second physiological sensor being adapted to generate a second physiological signal indicating a magnitude of a second physiological parameter of the user, the first RF signal further having a second binary parameter value representing the magnitude of the second physiological parameter of the user based on the second physiological signal.

5. The athletic performance monitoring display system of claim 4, wherein the flexible display screen of the flexible display device further includes a second display area disposed on the first side thereof, and the RF receiver of the flexible display device being further adapted to send the second binary parameter value to the first microprocessor, the first microprocessor further programmed to generate a third control signal to induce a graphical display object in the second display area to display the magnitude of the second physiological information based on the second binary parameter value.

6. The athletic performance monitoring display system of claim 1, wherein the monitoring device further includes an accelerometer sensor, the second microprocessor further operably coupled to the accelerometer sensor; the accelerometer sensor being adapted to generate an acceleration signal indicating an acceleration of the user, the first RF signal further having a second binary parameter value representing the acceleration of the user at the second time based on the acceleration signal.

7. The athletic performance monitoring display system of claim 6, wherein the flexible display screen of the flexible display device further includes a second display area disposed on the first side thereof, and the RF receiver of the flexible display device being further adapted to send the second binary parameter value therein to the first microprocessor, the first microprocessor further programmed to generate a third control signal to induce a graphical display object in the second display area to display the acceleration of the user based on the second binary parameter value.

8. The athletic performance monitoring display system of claim 7, wherein the flexible display screen of the flexible display device further includes a third display area disposed on the first side thereof, and the first microprocessor of the flexible display device further programmed to calculate at least one of a speed of the user and a distance traveled by the user based on the second binary parameter value, the first microprocessor further programmed to generate a fourth control signal to induce a graphical display object in the third display area to display at least one of the speed of the user and the distance traveled by the user.

9. The athletic performance monitoring display system of claim 1, wherein the monitoring device further includes a GPS sensor, the second microprocessor further operably coupled to the GPS sensor; the GPS sensor being adapted to receive position signals indicating first and second position coordinates of the user at second and third times, the first RF signal further having second and third binary parameter values representing the first and second position coordinates values, respectively.

10. The athletic performance monitoring display system of claim 9, wherein the flexible display screen of the flexible display device further includes second and third display areas disposed on the first side thereof, the RF receiver of the flexible display device being further adapted to send the second and third binary parameter values therein to the first microprocessor, the first microprocessor further programmed to calculate at least one of a distance traveled by the user, a speed of the user, and an acceleration of the user based on the second and third binary parameter values, the first microprocessor further programmed to generate a third control signal to induce a graphical display object in the second display area to display at least one of the distance traveled by the user, the speed of user, and the acceleration of the user.

11. The athletic performance monitoring display system of claim 1, wherein the graphical display object in the first display area comprises a bar graph having a plurality of rectangular segments disposed in a column and separate from one another, wherein a number of rectangular segments of the plurality of rectangular segments having a solid color representing the magnitude of the first physiological parameter at the first time.

12. The athletic performance monitoring display system of claim 1, wherein the first display area further includes a textual display area being adapted to display text identifying the first physiological parameter, and a decimal number indicating the magnitude of the first physiological parameter at the first time.

13. The athletic performance monitoring display system of claim 1, wherein the first physiological parameter comprises at least one of a heart rate, a breathing rate, and a skin temperature.

14. The athletic performance monitoring display system of claim 1, wherein the monitoring device further comprises a strap coupled to the housing, the ring-shaped strap adapted to attach the housing to a surface of a body of the user such that the first physiological sensor monitors the first physiological parameter of the user.

15. The athletic performance monitoring display system of claim 1, wherein the flexible display screen of the flexible display device further includes a second display area disposed on the first side thereof, the RF receiver of the flexible display device being further adapted to receive a second RF signal having video information, or static text information, or static graphical information therein, the first microprocessor programmed to generate a second control signal to induce the second display area to display the video information, or the static text information, or the static graphical information thereon.

16. A method for displaying information associated with a user, comprising:
attaching a flexible display device to the user, the flexible display device having a flexible display screen, a first microprocessor, an RF receiver, a flexible housing portion, and a first adhesive member; the flexible display screen having a first side, a second side, and a first disposed on the first side thereof; the first microprocessor operably coupled to the flexible display screen and the RF receiver; the flexible housing portion being coupled to the second side of the flexible display screen such that the first microprocessor and the RF receiver are disposed within an interior area defined by the second side of the flexible display screen and the flexible housing portion; and the first adhesive member being coupled to an external surface of the flexible housing portion and to the user;
attaching a monitoring device to the user, the monitoring device having a housing, a first physiological sensor, a second microprocessor, and an RF transmitter; the second microprocessor operably coupled to the first physiological sensor and the RF transmitter;
generating a first physiological signal indicating a magnitude of a first physiological parameter of the user at a first time utilizing the first physiological sensor;
generating a first control signal from the second microprocessor to induce the RF transmitter to transmit a first RF signal having a first binary parameter value representing the magnitude of the first physiological parameter of the user at the first time, based on the first physiological signal; and
receiving the first RF signal at the RF receiver of the flexible display device and sending the first binary parameter value to the first microprocessor;
generating a second control signal from the first microprocessor to induce a graphical display object in the first display area to display the magnitude of the first physiological parameter at the first time, based on the first binary parameter value.

17. The method of claim 16, wherein the monitoring device further includes an accelerometer sensor, the second microprocessor further operably coupled to the accelerometer sensor; the method further comprising:
generating an acceleration signal indicating an acceleration of the user at a second time utilizing the accelerometer sensor; and
generating a third control signal to induce the RF transmitter to transmit a second RF signal having a second binary parameter value based on the acceleration signal utilizing the second microprocessor, the second binary parameter value representing the acceleration of the user at the second time;

18. The method of claim 17, wherein the flexible display screen of the flexible display device further includes a second display area disposed on the first side thereof, the method further comprising:
receiving the second RF signal at the RF receiver of the flexible display device and sending the second binary parameter value therein to the first microprocessor; and
generating a fourth control signal utilizing the first microprocessor to induce a graphical display object in the second display area to display the acceleration at the second time based on the second binary parameter value.

19. The method of claim 16, wherein the monitoring device further includes a GPS sensor, the second microprocessor further operably coupled to the GPS sensor; the method further comprising:
receiving position signals utilizing the GPS sensor that indicate first and second position coordinates of the user at second and third times; and
generating a third control signal at the second microprocessor to induce the RF transmitter to transmit a second RF signal having second and third binary parameter values representing the first and second position coordinates values, respectively.

20. The method of claim 19, wherein the flexible display screen of the flexible display device further includes second and third display areas disposed on the first side thereof, the method further comprising:
receiving the second RF signal utilizing the RF receiver of the flexible display device and sending the second and third binary parameter values therein to the first microprocessor;
calculating a speed of the user and an acceleration of the user utilizing the first microprocessor based on the second and third binary parameter values;
generating a fourth control signal utilizing the first microprocessor to induce a graphical display object in the second display area to display the speed of user; and
generating a fifth control signal at the first microprocessor to induce a graphical display object in the third display area to display the acceleration of the user.
